(19) United States
(12) Patent Application Publication
(10) Pub. No.: US 2025/0246280 A1
(43) Pub. Date: Jul. 31, 2025

(54) AI-BASED HOME HEALTH SYSTEM AND METHODS OF USE

(71) Applicant: Diana Nelsen, Bristow, VA (US)

(72) Inventor: Diana Nelsen, Bristow, VA (US)

(21) Appl. No.: 18/736,448

(22) Filed: Jun. 6, 2024

Related U.S. Application Data
(60) Provisional application No. 63/625,286, filed on Jan. 26, 2024.

(51) Int. Cl. G16H 20/00; G16H 10/60; G16H 40/67; G16H 50/20

(52) U.S. Cl. CPC G16H 20/00; G16H 10/60; G16H 40/67; G16H 50/20

(57) ABSTRACT

An artificial intelligence based home health system. The system includes a setup module to set up the Initial requirements of the system, a monitoring module to monitor at least one condition or function of at least one user, a treatment module configured to assist in carrying out basic treatment not requiring medical facilities or a prescription, and an immediate treatment module configured to become alerted when the user needs immediate medical treatment.

### CROSS REFERENCE TO RELATED APPLICATION(S)

This application claims the benefit of, U.S. Provisional Application No. 63/625,286 entitled “AI-BASED HOME HEALTH-BASED SYSTEM AND METHOD OF USE”, filed on Jan. 26, 2024. The subject matter of this application is hereby incorporated by reference in its entirety

### TECHNICAL FIELD OF THE INVENTION

The present invention relates to the general field of healthcare, and specifically to systems for monitoring, providing alerts, diagnoses, and treatment recommendations outside a medical setting.

### BACKGROUND

Though the current U.S. healthcare system has advantages, Including technology and innovation, there are a number of reasons a patient may feel more comfortable avoiding the medical establishment when possible, finding health solutions at home, and treating themselves for at least minor issues at home, rather than seeking a medical professional. Because there is a doctor shortage in the U.S., seeking care is often an inefficient, frustrating process, involving long waits and high costs.

For those within a typical national medical system, whether a private or government health care system, there are definitely issues with room for improvement. For one, individuals or patients wanting to be seen by a medical professional must make an appointment to see their healthcare provider for assessment and treatment of symptoms, physicals, check-ups, etc. Then the patient has to work out transportation and other issues, such as someone staying with any children or pets. The patient travels to the medical professional, then is forced to wait to be seen, in often less than ideal conditions.

It is to be understood that herein, the term “medical professional” means a doctor, nurse practitioner, clinician, or anyone with at least some medical training that people tend to see for medical attention of some kind.

It is also to be understood that the term “user” or “patient” refers to any person or party who seeks or uses medical care, monitoring, information, or other medical assistance at any level. A patient can additionally be anyone who uses the system herein for themselves. A user can additionally be anyone or any party who uses the system herein for themselves or another party.

It is particularly difficult or inconvenient for patients who are homebound or otherwise incapacitated to arrive for appointments, then have to wait. Those seeking medical attention, by the nature of the circumstance, are often sick or in a weakened state. Nobody wants to spend hours waiting on uncomfortable chairs to see someone when they are already sick or injured. Waiting uncomfortably in a medical establishment can exacerbate their conditions, or at the least, make them even more reluctant to seek care than typical patients.

Further, given the prevalence of conditions like Hospital Acquired Infections (HAIs), (e.g., Methicillin-resistant Staphylococcus aureus (MRSA), forms of pneumonia, or other bacterial or viral infections), going to a medical facility, even a doctor's office, can often increase the danger for patients, and ironically, steering clear of medical facilities can improve a patient's chances of avoiding medical issues. Hospitals and other care centers, because these are the places people go when infected, are known to struggle with cleaning bacteria, and particularly viruses, from their premises. Particularly in a time when people are more mindful of the dangers of pandemics than ever, and more specifically the presence of viruses and bacteria within hospitals, many people have good medical reason to avoid medical facilities.

However, simply avoiding professional medical care comes with its own set of dangers. A patient's complaint or symptom is often a waring sign of something more serious. The problems of not having acute or painful conditions checked and treated are obvious. Without monitoring, serious conditions may be present and go undiagnosed, or even un-noticed until it is too late. What would have been minor issues can quickly grow into major issues. Unfortunately, this frequently happens, as people often do not have access to proper monitoring and check-ups, because of high costs, time, or other reasons.

Lack of access to, or a lack of seeking out, routine medical care can have a number of other unfortunate consequences. These can include delayed or missed diagnoses. Without timely access to healthcare providers, individuals may experience delays in receiving an accurate diagnosis of symptoms indicating health conditions. This can lead to prolonged suffering, worsening of symptoms, and potential complications. In some cases, missed or delayed diagnoses can even result in life-threatening situations.

Consequences can also include progression of illnesses. Without timely and prompt medical attention, conditions, diagnosed or not, can worsen, and individuals may experience a faster progression of their diseases or other conditions. For example, a minor infection left untreated can develop into a more severe and complicated infection.

Delayed access to healthcare providers can also result in reduced treatment options. Time lost may limit the available treatment options for patients. Some conditions are typically more treatable or manageable in the early stages, so that delaying treatment may reduce the effectiveness of interventions. In some cases, limited treatment options can lead to more invasive or costly procedures down the line.

There can also be increased healthcare costs. When individuals do not have timely access to healthcare providers, if their condition worsens through delayed or less monitoring or other care, this can lead to more complex medical needs. This can result in increased healthcare costs, as more extensive treatments or interventions may be required. Additionally, individuals may resort to emergency care, which is often more expensive than preventive or early Intervention measures.

Further, adverse health outcomes may result. Lack of timely access to medical providers and healthcare can also contribute to poorer health outcomes. Individuals may experience prolonged suffering, decreased quality of life, and increased risk of complications or disability. For some conditions, timely intervention can significantly improve the chances of successful treatment and recovery.

With disparate and unequal healthcare, there are often disparate and unequal healthcare results. Limited access to healthcare can result in disproportionate effects on marginalized and underserved populations. Individuals who lack timely access may be from low-income backgrounds, rural areas with limited healthcare infrastructure, minority communities, or they may have limited health insurance coverage. This can further deepen inequalities in health outcomes and perpetuate systemic disparities.

Besides the physical consequences, the inability to access healthcare providers in a timely manner can lead to psychological distress. Individuals may experience increased anxiety, fear, and frustration due to uncertainty about their health and difficulty of accessing care. Further, the stress of navigating healthcare systems without adequate support can also harm mental well-being.

Obviously, the consequences of delayed access to medical providers can vary depending on the individual, the specific condition, individual circumstances, and available alternatives. However, overall, timely access to healthcare providers is clearly better than delayed access to healthcare for catching problems early, promoting optimal health, preventing complications, and improving individual outcomes.

There have been some efforts to help provide some data collection and monitoring of patients, typically in healthcare settings, but these have been limited and only address a small part of the under-serviced patient problem.

Generally, collecting data from a patient and using computers to compile Information to create an information profile of the patient—basically computerization of the manual process of keeping files and records—is known in the art.

Additionally, collecting data and using a computerized process to provide a treatment recommendation based on the collected patient information—basically a computerized version of manually gathering patient and symptom information and recommending treatment based on that information—is likewise known in the art.

While these limited steps of computerizing portions of the process provide some enhancement of care at medical facilities, none of this solves any of the issues noted herein. None of these provide a path forward for effective, convenient, or reasonably safe at-home treatment options for patients.

For an example, some individuals have been able to take manual blood pressure at home and track the readings on paper. This provides some crude monitoring of a single factor, which can provide a general alert if there are large-scale changes. In a slight technological upgrade, some patients can go to some pharmacies and have their blood pressure taken by a machine. However, results from these machines, like paper readings, only give a single crude metric. This may alert a patient to a generalized problem needing checking, but it relies on access to such commercial machines, people using them regularly, the results being sufficiently large to raise concern, and people recognizing they need to follow through with a medical professional.

Accordingly, such computerized medical smart devices do not address the problems herein, and still require a patient to have to go to a medical facility and see a medical professional for even general monitoring needs. There are a number of further shortcomings.

First, a constant weakness of any AI system is that it is only as smart as its programming. Human judgment, experience, and the ability to consider contextual factors are crucial in making accurate diagnoses and treatment decisions. Such AI systems, though, are typically not able to understand context or think outside of their limited programmed logic. Such systems have a lack of human judgment and context, and may struggle to incorporate these elements effectively. Accordingly, AI systems can fall to capture the complexity and nuances of Individual cases.

Further, such systems, particularly within a medical context, are of limited scope and accuracy. AI devices rely on the data and algorithms they are trained on. If the training data is incomplete, biased, or does not represent the full range of conditions and symptoms, the accuracy of the device's diagnoses can be accordingly compromised.

These systems also can result in a lack of patient-provider interaction. Building a trusting patient-provider relationship, empathy, and communication are vital aspects of healthcare that cannot be fully replicated by an AI device alone. This can lead to a lack of human interaction and personalized care.

Current systems can also result in a limited ability to handle complex cases. Computerized systems often tend to focus best on straight lines between a single symptom and a single cause. Some medical conditions require a comprehensive and multidisciplinary approach for accurate diagnosis and treatment. AI devices may struggle with complex cases that involve multiple symptoms, and co-morbidities, or require the integration of various medical specialties. In such cases, the expertise and collaboration of healthcare professionals, and knowing when to arrange such collaborations, can be essential.

Accordingly, there is a thus-far unmet need for individual patients to receive individualized medical care that provides convenience, safety, choices of medical pathways, but also access, when needed, to medical professionals and facilities. Patients need a system in which they receive routine monitoring and treatment of minor symptoms or problems without having to make appointments with, and repeatedly come in and out of, medical facilities. However, they also need access to medical professionals and higher levels of care when needed and a system that can know when patients need what kind of care.

### SUMMARY

A patient-centered AI-based evaluative and care system, with a focus on home health. This individualized health system provides a way for users to receive convenient monitoring, guidance, and professional medical care—and when needed, access to contextual human judgment. The system is designed, generally, to maximize the role of home healthcare and the control of a patient and/or caretakers in a patient's care, while addressing safety concerns regarding reducing involvement by the medical establishment.

The system is a device-dependent machine-intelligent system, via one or more computerized devices, that uses algorithms and protocols to detect symptoms, and uses artificial intelligence techniques, such as machine learning or deep learning, to analyze various patient data sources and detect patterns or indicators of specific symptoms or medical conditions. This system will allow an individual user or patient to find solutions and treatments to a variety of health problems from home; if a possible treatment is sufficient to the condition, or if more immediate treatment is needed, it will alert the patient and/or medical professionals.

The system is generally comprised of system setup module, at least one computerized device(s), choosing a medical path, a monitoring module of steps and equipment to carry out monitoring activities, a treatment module of steps and equipment to carry out basic treatment, and an immediate treatment module of steps and equipment to carry out immediate treatment.

The system setup module provides for setting up the initial requirements of the system. In a preliminary step, at least one computerized device is provided, and system software is pre-loaded into the at least one computerized device.

The at least one computerized device can be comprised of at least one of any suitable computerized device known in the art, which can include, e.g., a server, personal computer, smartphone, or tablet. Further, the at least one computerized device can be comprised of, in a number of embodiments, varying numbers, forms, and structures.

A patient or other appropriate party can make a decision whether to seek an allopathic or homeopathic path of treatment. The patient/user can make this decision at any chosen workable time, such as during system setup, another point before the monitoring module, or even at a later point before treatment.

If an allopathic treatment is selected, the system will provide a patient with a list of non-prescription treatment products to purchase from a pharmacy or other source. The system will also provide a recommended treatment plan for these product(s) and monitoring will continue until the user's condition improves. If a homeopathic path is chosen, the system will provide product(s) that can be obtained reasonably easily from a nutrition or health store or other source and will provide a suitable treatment plan to be implemented.

Whichever treatment path is being used, an important point is that the treatment products, administration, or other treatment actions are within the capability and reach of the individual user. The user, or someone in user's proximity, can obtain products for, and implement, the treatment.

The system can be set up and turned on by the patient or other user to monitor the patient. This can be done in a series of steps within a monitoring module. Within a monitoring phase, data for a patient profile is collected, assembled, and the patient profile created. The system can enter a continuous assessment phase of the patient, in which the patient is under constant monitoring and re-assessment by the system. The system can monitor a number of biological parameters of the patient during this continuous monitoring.

The system can, with continuous monitoring or routine interval monitoring, compare each successive profile to the built-up data profile within the computerized device of the system. Recursive assessment of the patient by the system can, over time, build a stronger and stronger data profile of the patient. If the parameters of one or more symptoms move too far outside the built-up profile, the system can flag these and make a decision whether the patient should treat the condition(s) within the treatment module or recommend the patient move to the immediate treatment module.

In an embodiment, a patient's data and current dentition are transferred into a first database which the system has immediate access to, on a constant or periodic basis, so this information is always there. Generally, the first database has a number of other patient profiles, protocol information, and any other appropriate information for comparison.

In another embodiment, the patient's data and current condition are transferred into the first database once a patient's monitored conditions are outside normal range (has symptom(s).

Within the monitoring module, the system, while monitoring a patient, receives incoming data, and the system can use AI to determine what level of care a patient needs and signal that determination. This determination can be signaled in a number of ways known in the art, such as audio signal, colored light, printout, or other method, but it is important that this determination be communicated in a way the patient, or any user working with the system, can easily notice and acknowledge.

Within the treatment module, there are generally a diagnostic phase and a treatment phase. In some embodiments, the diagnosis and treatment can be combined.

Once a patient's monitored conditions are outside normal range (i.e., has symptoms), the software can use this information that has been sent to the first database to create a specific query (i.e., information request) using AI.

In an alternative, if there is enough information such as other patient profiles within the first database to supply a diagnosis, the system can use AI to proceed with the diagnostic phase and treatment phases.

If the system uses the first database to create a specific query, the system can transmit the query for info (using AI) to the second, likely larger database, for further processing. The database can be a read-only database that is part of a larger system, such as, e.g., a large hospital network, a private data collection and processing network, or government system such as Medicare.

After the second database is pinged with the transmitted query, the at least one computerized device, via the second database, processes the query, gathering and copying a number of patient files and any other relevant information in the database and transmits the copied data back to a part of the system, in an embodiment to the first database. Another way to see this is that the at least one computerized device operating the second database, upon query, decides what to pull down and send to the first database. To process the query, the system, software operating the second database, or both, gathers a number of data files that are closest, or most relevant to, the query. This is typically a number of patient files. The number of files can be any suitable to provide a sufficient sample size.

The system now processes the file data by analyzing and comparing the data of the working copies of the most relevant files in the first database to the information in the patient file. Upon comparing the data, and particularly the symptoms, in the patient file to the downloaded files in the second database, a diagnostic list is provided. The diagnostic list is a list of possible causes of the patient's symptoms that can be discerned from the data—from most probable down to possibilities with very small probability. Each of the diagnostic possibilities on the list can also include detailed reasons for its presence on the list. The detailed reasons can help with evaluation and treatment further in the process.

With a list of diagnoses supplied, the treatment phase can be entered. This list can be used by the system to create a model that determines a treatment plan, typically also using AI. One or more processing databases can be used here, along with the at least one computerized device, to process and implement the treatment plan. The treatment plan can provide a systematic approach, making use of the diagnostic list in order of likelihood, so that the more likely possibilities are treated for first, maximizing chances of successful treatment as early as possible within the treatment phase. Further, to increase efficiency, the system can decide whether multiple possibilities can be treated for at the same time, increasing the likely speed of successful treatment.

As the treatment is being administered within the standard treatment module, the system can continually monitor the patient to see if the symptom(s), signs, and/or overall patient condition are moving closer to the standard profile of the patient. If the patient condition is moving closer to their standard profile, the patient is likely improving. If the patient condition is moving further from their standard profile, the patient condition is likely becoming worse. When the indicators of patient's symptoms/condition move back within an acceptable range, the system will move the patient back to standard monitoring. If the symptom or condition indicators become sufficiently out of range, the system will likely determine that the user needs more immediate medical care than can be provided by them, and the user must seek more immediate, acute care.

Accordingly, if a patient has non-life-threatening symptoms, a good amount of testing, information gathering, diagnosis, and treatment can be done from the comfort of the patient's own home, within hours or perhaps seconds.

If anywhere in the previous steps, the system determines that immediate treatment is needed, the system moves the patient into the immediate treatment module. Depending upon the condition of the patient, the system can recommend a specific type of medical treatment. Depending upon factors such as the certainty of need, the severity of the condition, symptom(s), and the immediacy of the need for treatment, the system may contact a medical professional for guidance and judgment. Or the system may make an appointment for the patient to be seen by an appropriate professional. Or if necessary, the system may call for emergency assistance immediately, including calling out to 911.

This system maximizes monitoring of a patient and effective treatment of a condition, while providing maximum convenience and assurance to the patient or user that acute conditions will receive the proper notice and treatment and making that patient or user aware when conditions are such that the medical establishment does need to be contacted. This provides treatment that comes with peace of mind for the patient and/or user.

The system can be placed within a number of monitoring and treatment configurations, with chosen monitoring system.

The system can, overall, provide reassurance and peace of mind for users, as well as caregivers and family members who are responsible for monitoring the health of their patients or loved ones. The system can offer continuous monitoring and alerts, enabling caregivers to respond quickly in case of emergencies or changes in health status.

A recursive assessment phase can also be added to the process of the system. In this phase, the AI of the system can compare data such as treatment and results data from the patient, to other iterations of the same data for the same patient, to data from other patient files, or both. Over a number of comparisons and processing by the AI, predictions, treatments, and results can be recursively improved over time. The more data is added, especially with machine learning, the more monitoring, diagnosis, and treatment plans for the patient will improve going forward.

In another embodiment, an interactive audio/visual module can also be added to the system. The interactive module can assist with the safety, for example, of a resident living at home. The module can be comprised of detection equipment for monitoring a residence. The equipment can be audio detection equipment, visual, vibration, or motion detecting equipment, or other suitable equipment known in the art for the purpose herein.

The equipment is installed and monitored by the system. If a sufficient change is detected by the system indicating a possible problem, it can become alerted. It can alert friends, relatives, a doctor's office, or call emergency services, or take other appropriate action.

### BRIEF DESCRIPTION OF THE DRAWINGS

FIG. 1 is a schematic diagram of an embodiment of the invention.

FIG. 2 is a schematic diagram of an alternative embodiment of a portion of the invention.

FIG. 2a is a schematic diagram of another alternative embodiment of a portion of the invention concerning computerization.

FIG. 2b is a schematic diagram of another alternative embodiment of a portion of the Invention concerning computerization.

FIG. 3 is a schematic diagram providing further detail of a portion of the embodiment of FIG. 1.

FIG. 4 is a schematic diagram providing further detail of a portion of the embodiment of FIG. 1.

FIG. 5 is a schematic diagram providing further detail of a portion of the embodiment of FIG. 1.

FIG. 6 is a schematic partially perspective view of an embodiment of a portion of the Invention in operation.

FIG. 6a Is a schematic front view of another embodiment of a portion of the invention in operation.

FIG. 6b is a schematic front view of another embodiment of a portion of the invention in operation.

FIG. 6c is a schematic front view of another embodiment of a portion of the invention in operation.

FIG. 7a-7b is a schematic diagram showing a possible specific embodiment of the invention.

FIG. 8 is a schematic diagram featuring a possible portion of the invention.

FIG. 9 is a schematic diagram featuring a possible portion of the Invention.

### DETAILED DESCRIPTION OF PREFERRED EMBODIMENTS

Shown and described herein is a patient-centered AI-based evaluative and care system, with a focus on home health, as well as a method of providing and a method of use. The individualized health system 2 herein provides a way for patients to receive convenient monitoring, guidance, and professional medical care—and when needed, access to contextual human judgment.

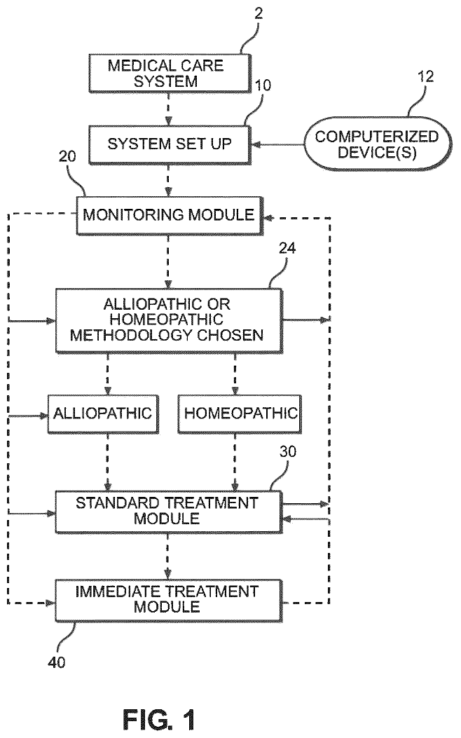

Turning to FIG. 1, an embodiment of the system 2 is disclosed. The system 2 is designed, generally, to maximize the role of home healthcare and the control of a patient and/or caretakers in a patient's care, while addressing safety concerns regarding reducing involvement by the medical establishment.

The system 2 is a device-dependent machine-intelligent system, via one or more computerized devices, that uses algorithms and protocols to detect symptoms, and uses artificial intelligence techniques, such as machine learning or deep learning, to analyze various patient data sources and detect patterns or indicators of specific symptoms or medical conditions. This system 2 will allow an individual user or patient to find solutions and treatments to a variety of health problems from home; if a possible treatment is sufficient to the condition, or if more immediate treatment is needed, it will alert the patient and/or medical professionals.

Herein, a user can be defined as one or more patients using the system, another person, party or entity, such as someone who has been appointed or has medical power of attorney, a medical professional, or other appropriate party.

The system 2 is generally comprised of system setup module 10, at least one computerized device(s) 12, choosing a medical path 24, a monitoring module 20 of steps and equipment to carry out monitoring activities, a treatment module 30 of steps and equipment to carry out basic treatment, and an immediate treatment module 40 of steps and equipment to carry out immediate treatment.

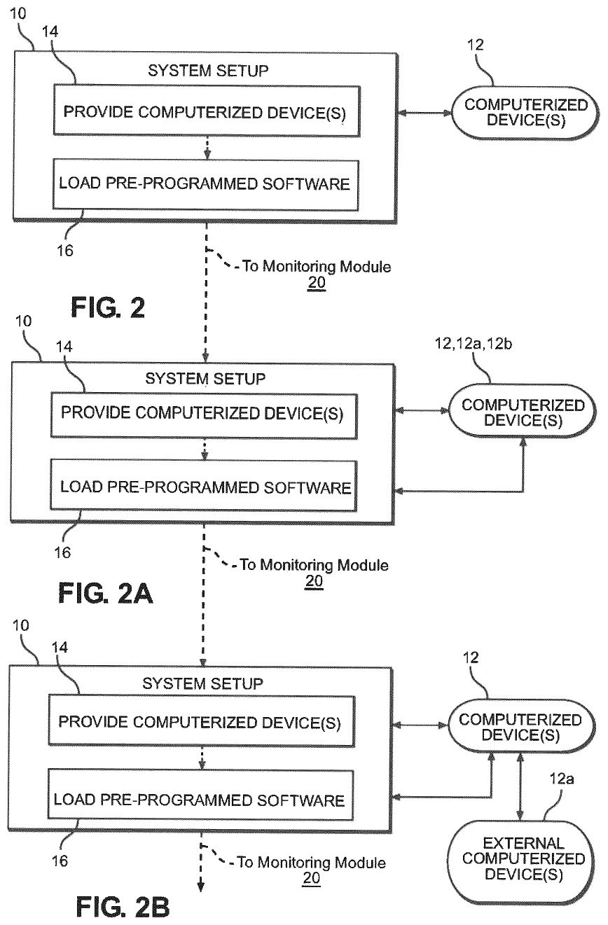

Turning to FIGS. 1 and 2, the system setup module 10 provides for setting up the initial requirements of the system. In a preliminary step, the at least one computerized device 12 is provided 14. System software is pre-loaded 16 into the at least one computerized device 12. The pre-programmed software is capable of performing the method embodiments herein.

Turning to FIGS. 1-5, and particularly FIG. 2, as indicated by the dashed arrows, some or all of the steps of the methods herein can be completed, or completed in a modified order, to represent a number of embodiments of the invention herein.

Turning briefly to FIGS. 2-2B, the at least one computerized device 12 can be comprised of at least one of any suitable computerized device known in the art, which can include, e.g., a server, personal computer, smartphone, or tablet. Further, the at least one computerized device 12 can be comprised of, in a number of embodiments, varying numbers, forms, and structures. A few representative configurations of the at least one computerized device 12, 12a, 12b, are shown in FIGS. 2-2b.

Turning to FIG. 2, in one embodiment, the at least one computerized device 12 can be comprised of a server computer. The software would be added to this computer and a user would access this computer directly.

Turning to FIG. 2a, the at least one computer can be comprised of a network of computers, represented here as 12, 12a, and 12b. The software can be loaded 16 into some or all of the computers 12, 12a, 12b, and the user may be able to access and use one, some, or all of the computers 12, 12a, 12b.

Turning to FIG. 2b, in another representative embodiment, one computer 12 can be a server and the second computerized device 12a can interact with each other. Data can be entered into the server, or data can be entered into the second computer 12a such as testing data, and the data be transferred from one computerized device to the other. Also, the second computerized device 12a can access and use the software on the server computer 12, or both computers 12, 12a may independently have the pre-programmed software. When the second computerized device 12a finishes a task, it can load the finished work product onto the server 12.

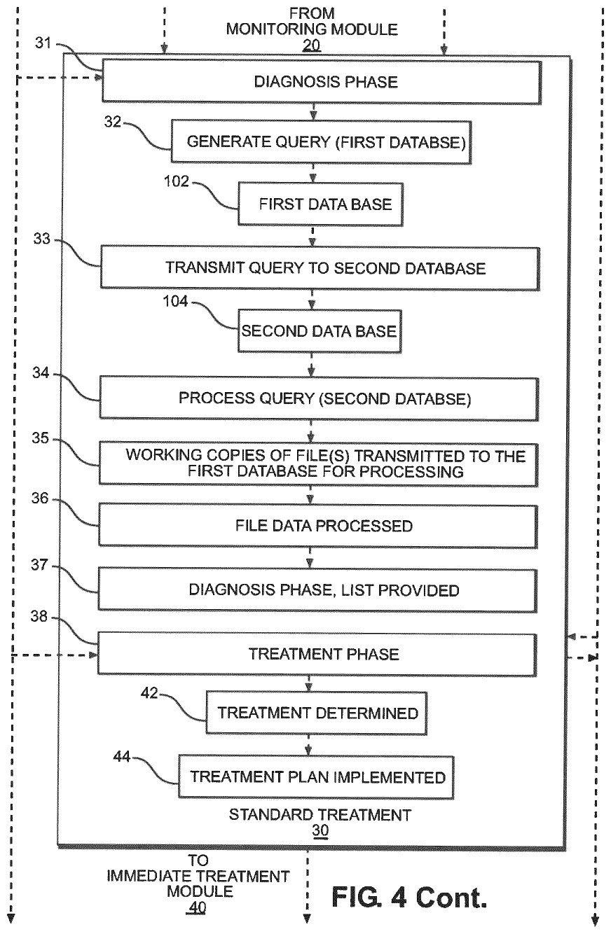

Turning to FIG. 4, the patient or other appropriate party can make a decision whether to seek an allopathic or homeopathic path of treatment 24. In this embodiment, the decision is made after the monitoring module 20. However, the patient/user can make this decision at any chosen workable time, such as during system setup 10, another point before the monitoring module 20, or even at a later point before treatment.

Whatever point in the process is chosen, the user/patient chooses an allopathic system or homeopathic system 24 for monitoring and treatment. Generally, an allopathic system of medicine (i.e., “standard medicine” or “western medicine”) relies on the use of pharmacological drugs and other physical Interventions to either treat or suppress symptoms, diseases, and health conditions. It often relies upon suppression of physical symptoms while the underlying condition is treated.

A homeopathic system of medicine, by contrast, encourages a more holistic approach, providing healing and wellness by examining and treating patient's overall conditions and the root cause of the illness, rather just treating the symptoms. Homeopathic treatments tend to rely on the premise that a patient's overall conditions, such as nutrition, overall health, living condition and other overall factors, play a role in the symptom or disease.

The system 2 can also recommend an allopathic treatment or a homeopathic treatment, based on factors like the data provided and user preferences.

If an allopathic treatment is selected, the system 2 will provide a patient with a list of non-prescription treatment products to purchase from a pharmacy or other source. These are off-the-shelf product(s) that are reasonably easy or easier to obtain and use. The system 2 will also provide a recommended treatment plan for these product(s) and monitoring will continue until the user's condition Improves. If a homeopathic path is chosen, the system 2 will provide product(s) that can be obtained reasonably easily from a nutrition or health store or other source and will provide a suitable treatment plan to be implemented. These products can include, for example, vitamins, natural substances or the like, or a combination.

Whichever treatment path is being used, an important point is that the treatment products, administration, or other treatment actions are within the capability and reach of the Individual user. The user, or someone in user's proximity, can obtain products for, and implement, the treatment.

Further, whichever treatment option is selected, the user can, if the results are not what is desired, select the other option and begin treatment again. For example, if the user chooses an allopathic treatment and the results are not sufficient, the user can go back, select a homeopathic treatment, and try that. Or vice versa.

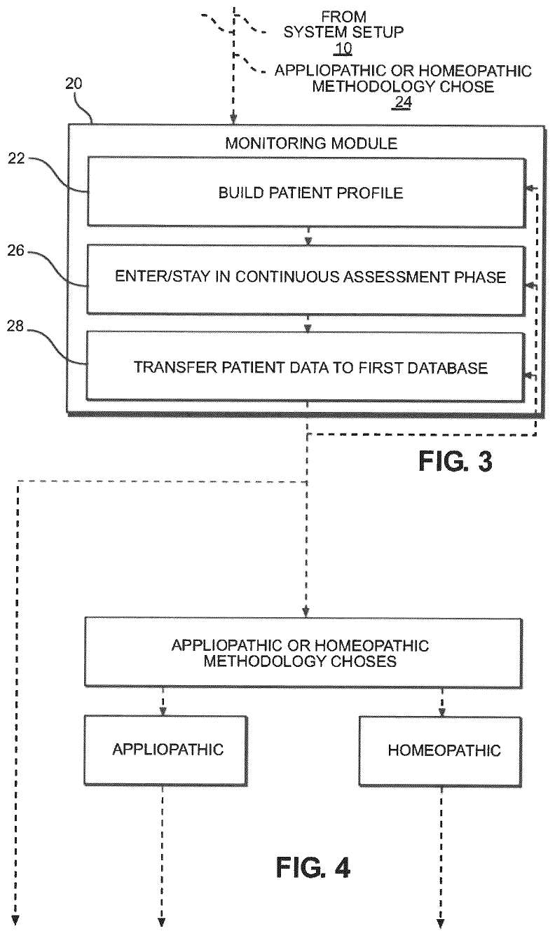

Turning to FIG. 1, and particularly to FIG. 3, as per the addition of the pre-programmed software 16, the system 2 can be set up and turned on by the patient or other user to monitor the patient. This can be done in a series of steps within a monitoring module 20. Within the monitoring module 20, data for a patient profile is collected, assembled, and the patient profile created 22. As known in the art, a patient record/file is assembled with patient data and turned into a profile 22. The patient profile 22 can include a comprehensive collection of structured and unstructured data that provides a holistic view of an individual's current health state and medical history. Collected patient profile information can include, but Is not limited to:
Demographic Information
Medical History
Medication History
Allergy and Adverse Reaction Information-Diagnostic Test Results
Treatment Plans and Procedures
Care Team Information
Care Coordination and Communication
Secured Sensitive and Confidential Information

The patient profile of the user can be shown under normal conditions when the patient has no symptoms. Then the system 2 can enter a continuous assessment phase 26 of the patient, in which the patient is under constant monitoring and re-assessment by the system 2.

The system 2 can monitor a number of these parameters of the patient during this continuous monitoring 26. For example, the system 2 can monitor patient respiratory parameters such as respiratory rate, and strength of breath, temperature parameters such as temperature or level of fever, heart parameters such as reported chest pain, swelling, numbness, palpitations, and other biological parameters such as blood pressure, dizziness, or headaches. A number of baseline parameters can further be included within the profile, such as weight, cholesterol, iron level, heartbeat, pulse, and any other appropriate conditions that can be checked or monitored.

Some of these parameters (e.g., blood pressure, temperature, or pulse) can be monitored by routine Interaction between the patient and the system 2. Others, such as high phlegm, dizziness, or headache, have to be self-reported by the patient. The system 2 can be programmed to routinely ask a user a list of questions.

The system 2 can, with continuous monitoring 26 or routine interval monitoring (for example, once a week or once a month), compare each successive profile to the built-up data profile within the computerized device 12 of the system 2. Recursive assessment of the patient by the system 2 can, over time, build a stronger and stronger data profile of the patient. If the parameters of one or more symptoms move too far outside the built-up profile—for example, the user's temperature and blood pressure are both too high—the system 2 can flag these and make a decision whether the patient should treat the condition(s) within the treatment module 30 or recommend the patient move to the immediate treatment module 40.

Turning briefly to FIGS. 3 and 4, in an embodiment, a patient's data and current dentition are transferred 28 into a first database 102 which the system 2 has immediate access to, on a constant or periodic basis, so this information is always there. Generally, the first database 102 has a number of other patient profiles, protocol information, and any other appropriate information for comparison.

In another embodiment, the patient's data and current condition are transferred 28 into the first database 102 once a patient's monitored conditions are outside normal range (has symptom(s).

Returning to FIG. 3, within the monitoring module 20, the system 2, while monitoring a patient, receives incoming data, and the system 2 can use AI to determine what level of care a patient needs and signal that determination. This determination can be signaled in a number of ways known in the art, such as audio signal, colored light, printout, or other method, but it is important that this determination be communicated in a way the patient, or any user working with the system, can easily notice and acknowledge.

In this embodiment, this determination of care needed is communicated with a color-coded signal. There are a number of color schemes that can be employed.

In some embodiments, if the patient has no indicated conditions, this may be indicated with a neutral or calming color outside the red-green-yellow set (such as, e.g., white, blue, or purple), or no color. If there is a condition mild enough to be referred on to the standard treatment module 30 and treated by non-prescription medicines or methods, it is indicated with a green color.

If the condition is concerning enough that careful monitoring or a doctor or urgent care visit is Indicated, this may be referred into the immediate treatment module 40 with a yellow indicator, meaning that contacting a medical professional is important and should be done soon, but is not urgent. An urgent condition needing Immediate treatment, such as a 911 call and immediate transport to a medical professional, can be signaled in red.

In other embodiments, if there are no conditions, this may be indicated with a green signal. If there is a condition mild enough to be referred on to the standard treatment module 30, and treated by non-prescription medicines or methods, it can be indicated with a yellow color.

If the condition is either concerning enough that careful monitoring or a doctor or urgent care visit is Indicated, or there is an urgent condition needing immediate treatment, such as a 911 call and Immediate transport to a medical professional, either of these may be referred into the immediate treatment module 40 with a red indicator.

Turning to FIG. 4, within the treatment module 30, there are generally a diagnostic phase 31 and a treatment phase 38. In some embodiments, the diagnosis and treatment can be combined.

Once a patient's monitored conditions are outside normal range (i.e., has symptoms), the software can use this Information that has been sent to the first database 102 to create a specific query (i.e., information request) 32 using AI.

In an alternative, if there is enough information such as other patient profiles within the first database 102 to supply a diagnosis, the system 2 can use AI to proceed with the diagnostic phase 31 and treatment 38 phases.

If the system 2 uses the first database 102 to create a specific query 32, the system 2 can transmit the query for info 33 (using AI) to the second, likely larger database 104, for further processing. The database 104 can be a read-only database that is part of a larger system, such as, e.g., a large hospital network, a private data collection and processing network, or government system such as Medicare.

The AI of the system 2, in some embodiments, can communicate in both directions, between the system 2, first database 102, and second larger database 104.

After the second database 104 is pinged with the transmitted query 33, the at least one computerized device 12, via the second database 104 (with possibly a further computerized device(s) operating the second database 104), processes the query 34, gathering and copying a number of patient files and any other relevant information in the database 104 and transmits the copied data 35 back to a part of the system 2, in this embodiment to the first database 102. Another way to see this is that the at least one computerized device 12 operating the second database 104, upon query 33, decides what to pull down 34 and send 35 to the first database 102. To process the query 33, the system 2, software operating the second database 104, or both, gathers a number of data files that are closest, or most relevant to, the query. This is typically a number of patient files. The number of files can be any suitable to provide a sufficient sample size. This could be any appropriate number such as 1,000, 10 million, or another number.

The system 2 now processes the file data 36 by analyzing and comparing the data of the working copies of the most relevant files in the first database 102 to the information in the patient file. Upon comparing the data, and particularly the symptoms, in the patient file to the downloaded files in the second database 104, a diagnostic list is provided 37. The diagnostic list is a list of possible causes of the patient's symptoms that can be discerned from the data—from most probable down to possibilities with very small probability.

For example, a set of symptoms the system 2 has determined the patient has is processed 36 by comparing them with similar symptoms in a number of other patient files. The causes of the symptoms in these other patient cases are generally known. With these specific symptoms, the diagnostic list of possible causes is generated 37. In a hypothetical example, the list might read as follows:

| | | |
|---|---|---|
| Possible Cause 1 | 71% | probability |
| Possible Cause 2 | 22% | probability |
| Possible Cause 3 | 5% | probability |
| Possible Cause 4 | 1.9% | probability |
| Possible Cause 5 | 0.06% | probability |
| Possible Cause 6 | 0.03% | probability |

These probable causes together add up to 99.99% total probability that the condition is one of these. While this is not 100% of the possibilities, it's very close; and arguably more importantly, it is closer than human diagnosis would likely come. Each of the diagnostic possibilities on the list can also include detailed reasons for its presence on the list. The detailed reasons can help with evaluation and treatment further in the process.

With a list of diagnoses supplied 37, the treatment phase can be entered 38. This list can be used by the system 2 to create a model that determines a treatment plan 42, typically also using AI. One or more processing databases can be used here, along with the at least one computerized device 12, to process and implement the treatment plan 44. The treatment plan can provide a systematic approach, making use of the diagnostic list in order of likelihood, so that the more likely possibilities are treated for first, maximizing chances of successful treatment as early as possible within the treatment phase 38. Further, to increase efficiency, the system 2 can decide whether multiple possibilities can be treated for at the same time, increasing the likely speed of successful treatment.

Referring back to the example diagnostic list, the system may determine that the treatments for Causes 1 and 2 would conflict with each other, but that Cause 1 and 3-6 can be treated for simultaneously, so that all but the 22% likelihood of Cause 2 is treated for at once. If the treatment of about 78% of likely causes is not successful, then Cause 2, the remaining 22% can be treated for in a second round.

In another embodiment, there can be additional databases or different division between databases. For example, there can be additional databases to the first and/or second database, 102, 104, or a database can be comprised of two subsidiary databases that interact with each other.

In other embodiments, human review can also be included, such as within the diagnostic phase 31, and before the treatment phase 38 is begun. After the system 2 makes a recommendation for a treatment plan 42, one or more human medical reviewers can look over the patient Information, diagnostic list, and recommended treatment plan to determine if the treatment plan is congruent with their medical expertise. The human medical review element can be a doctor, LPN, medical team, or other appropriate parties. There can also be additional AI level of review. The AI performing this review may be taught (i.e., machine learning) through the experiences of the human review.

The human medical review element can review the processes, do duplicate or additional tests, and approve or object to the treatment plan. This review can provide patients with an extra level of assurance that the treatment plan supplied by the system 2 is congruent with known Information and medical understanding. If a patient has entered the immediate treatment phase 40, the medical review personnel can, in addition, run recommended tests for diagnosis and provide the treatment themselves.

As the treatment is being administered within the standard treatment module 30, the system 2 can continually monitor the patient to see if the symptom(s), signs, and/or overall patient condition are moving closer to the standard profile of the patient. If the patient condition is moving closer to their standard profile, the patient is likely improving. If the patient condition is moving further from their standard profile, the patient condition is likely becoming worse. When the indicators of patient's symptoms/condition move back within an acceptable range, the system 2 will move the patient back to standard monitoring 20. If the symptom or condition indicators become sufficiently out of range, the system 2 will likely determine that the user needs more immediate medical care than can be provided by them, and the user must seek more immediate, acute care.

Accordingly, if a patient has non-life-threatening symptoms, a good amount of testing, information gathering, diagnosis, and treatment can be done from the comfort of the patient's own home, within hours or perhaps seconds.

This system 2 offers a vast improvement over the current state of care, in which a patient often has to make an appointment with a doctor or other medical facility, which itself can take weeks, get a “most likely” possible diagnosis, try the treatment for that for weeks or months, then if unsuccessful, make another appointment, return, get another possible diagnosis, and attempt the treatment for that. The system can save a patient frustrating, costly weeks or even months of running in and out of doctor's offices, hospitals, or other facilities.

In addition, it can also supply information to, and work with specialists for, better overall outcomes regarding known conditions. For example, many specialist doctors prescribe IV home infusion therapy and IV antibiotic therapy at home for their patients. The system 2 can provide accurate drug-level results in such situations to monitor whether the treatment is effective or If adjustments need to be made for the specialist doctor's patients at home. This can include Vitamin D levels, etc. This would be beneficial for both the physicians and the patients.

The system 2 can also work with medical professionals to supply patient information and speed up diagnoses and treatment without the patient needing to come in. As an example, the system 2 can enable patients to submit urine or blood samples for analysis, either on site or at a testing facility, for quick analysis. For instance, many elderly patients fear urinary tract infections (UTIs). A diagnosis could be rapidly established if the system 2 permits direct sample submission. This avoids the delay of transporting urine to the lab and awaiting a doctor's confirmation for antibiotic treatment of the UTI. Alternatively, a holistic approach could be considered, like using dandelion herb, which has disinfectant properties that inhibit bacterial growth in the urinary system.

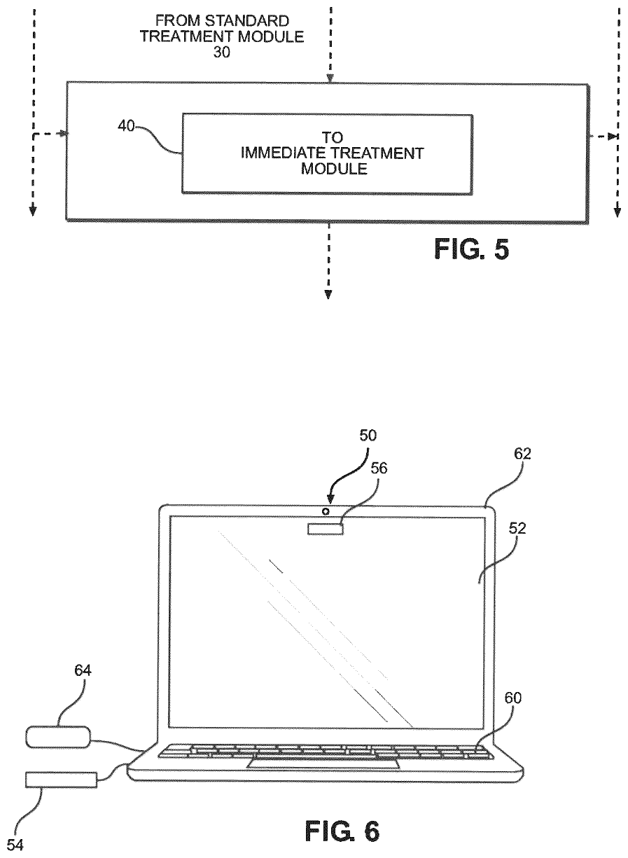

Turning to FIG. 5, if anywhere in the previous steps, the system 2 determines that immediate treatment is needed, the system 2 moves the patient Into the immediate treatment module 40. Depending upon the condition of the patient, the system can recommend a specific type of medical treatment. Depending upon factors such as the certainty of need, the severity of the condition, symptom(s), and the Immediacy of the need for treatment, the system 2 may contact a medical professional for guidance and Judgment. Or the system 2 may make an appointment for the patient to be seen by an appropriate professional. This may Include a general nurse practitioner, doctor, specialist, or other medical professional. Or if necessary, the system 2 may call for emergency assistance immediately, including calling out to 911.

This system 2 maximizes monitoring of a patient and effective treatment of a condition, while providing maximum convenience and assurance to the patient or user that acute conditions will receive the proper notice and treatment and making that patient or user aware when conditions are such that the medical establishment does need to be contacted. This provides treatment that comes with peace of mind for the patient and/or user.

### EXAMPLES OF USE

### Example 1

Bob has a large, skinned area on his leg that is somewhat painful but not threatening. Rather than go to an emergency room, pay possibly thousands of dollars, and wait for hours, Bob decides to treat it himself. The system 2 in this case can bypass the monitoring module 20, as this is a known injury. He consults the system 2, including explaining his problem and answering a few questions about symptoms. The system 2 can check Bob, his injury, and basic vital signs.

The system 2 enters the treatment module 30. The system 2 loads or accesses Bob's profile and enters the diagnosis phase 31. The system provides a diagnosis. This process is somewhat abbreviated as it is already known he has a skinned area, and the question is more about how severe it is. The system 2 then enters the treatment phase 38 in which the system 2 recommends a course of treatment involving allopathic medicine, holistic homeopathic treatment, or a combination. Bob gets and applies the recommended treatment and reports the results to the system 2 at a number of system-recommended time points. Bob may even take pictures of the treated area over time so the system 2 can-similar to a medical professional-scan and visually monitor progress.

The system 2 can be configured to report the Incident and symptoms to his doctor. The doctor can do a quick check and confirm treatment rather than have another in-person patient to see and more visit-related paperwork to fill out.

There is a color-coded component to monitor progress. If treatment progresses, the system stays in the “green.” Bob simply continues the recommended consumer treatment and the system 2 stays in standard treatment mode 30, until the condition clears.

If the condition fails to improve or seems to be getting slowly worse, the system 3 may go into “yellow” and enter the immediate treatment module 40. The system 2 recommends that Bob seeks medical treatment. In this way, the system assists Bob with use of home treatment by letting Bob know the point at which home treatment is not working and he needs to seek professional treatment. The system 2 can also notify Bob's doctor to arrange an appointment. This serves as an important backstop for home treatment.

If the system notices alarming symptoms or rapid worsening of condition, it can go “red” to immediate treatment 40. This lets Bob know he needs to seek immediate medical treatment. The system may also be configured to contact Bobs doctor, or if a more dire emergency, a nearby hospital or if Bob in unresponsive, contact 911 immediately. This can provide an important safety net for home treatment.

### Example 2

Joan's daughter, Kerry, has what appears to be a viral infection. Kerry feels under the weather, but Joan is not sure she needs to go to a medical center. Joan does not want to take her sick child to a medical center, where they are both likely to have to wait for hours to see someone. Plus, Joan is concerned about Kerry catching a virus at the medical center—the last thing Kerry needs while already sick. On the other hand, Joan doesn't want to be irresponsible and not take her daughter for professional-level medical care if she needs it.

Using the system 2, Joan enters Kerry's symptoms and issues (like in the example of Bob above). The system 2 follows up with any additional requests for information or data. The system 2 will then provide one or more possible diagnosis 37 in the diagnostic phase 31 and place Kerry in one of the three color-coded states for level of treatment, placing her in an appropriate treatment module 30 or 40 with a treatment plan in the treatment phase 38. If the system 2 recommends a home treatment option, Kerry is in “green” status and moves into standard treatment module 30. The system 2 will determine a treatment plan 42, with a course of home treatment based recommending over-the-counter items to use in treatment. These treatments can be allopathic, homeopathic, or a combination, depending on which of these paths is chosen 24.

The system 2 will continuously ask about Kerry's condition and collect information. If Kerry's condition gets worse, the system will change color code, and recommend accordingly.

If the symptoms indicate to the system 2 that professional medical care is indicated, it will go into a “yellow” status and move to the immediate treatment module 40 and recommend professional treatment. If the system 2 picks up signs of immediate emergency or rapidly declining condition, the system 2 will go into “red” within the immediate treatment mode 40 and recommend Joan take Kerry to a medical facility Immediately, or if extremely serious, contact 911.

In this example, Joan selects an allopathic course 24, the system 2 recommends a course of treatment, and Joan gets the over-the-counter medicine recommended and places Kerry on bed rest. Joan enters input regarding Kerry's condition into the system 2 periodically, or the system collects the information itself, and Kerry begins to get better. The system 2 stays in “green” within the standard treatment module 30 the whole time and Kerry recovers. Upon recovery, the system can move Kerry back into general monitoring mode 20. Over-the-counter and home treatment was adequate. Kerry had a virus and fought it off. Joan and Kerry were saved a trip to the hospital or doctor's office, which would have been of dubious value in a fight against a virus.

### Example 3

Renee is being monitored in monitoring module 20. One of the things the system 2 checks for is signs of skin cancer. An AI algorithm, with use of a visual scanner, trains on large datasets of skin images and provides real-time assessments for possible skin cancer. The system 2 spots possible signs of early skin cancer, moves into immediate treatment mode 40, changes its status to “yellow,” and refers Kerry to a dermatologist. Gathered early detection information from the system 2 is sent to the dermatologist and assists the dermatologist, who provides an early diagnosis. Renee is treated promptly and eventually returns to normal condition and returns to regular monitoring 20.

### Example 4

Mark is being monitored in monitoring module 20. One of the things the system 2 does is Diabetic Retinopathy screening. AI algorithms and the scanner 54 can analyze retinal images to detect signs of diabetic retinopathy, a complication of diabetes that affects the eye. The system 2 spots possible signs of early Diabetic Retinopathy and moves into immediate treatment mode 40, changes its status to “yellow,” and refers Mark to a doctor who can diagnose any problems. Gathered early detection Information from the system 2 assists the doctor who provides an early diagnosis. Mark is treated promptly and the problem is stopped.

### Example 5

The system is given data about Kylie, an infant, and monitors Kylie within the monitoring module 20, with periodic readings, profile building, and data gathering. The system 2 can keep track of Kylie's sleeping patterns, heart rate, surrounding temperature, and movements. There are no detected issues, and Kylie's parents are given continuous peace of mind by the system 2.

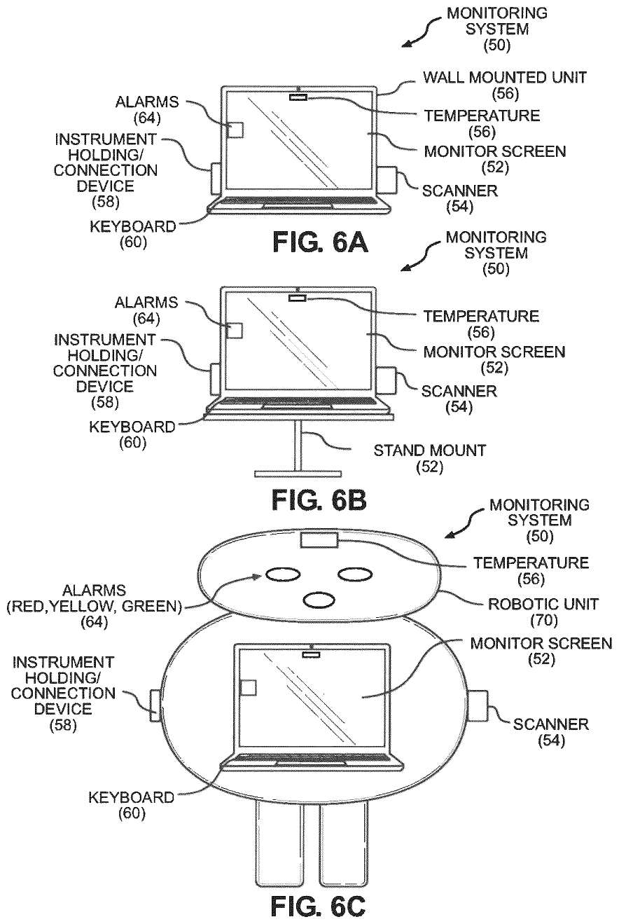

Turning to FIGS. 6-6c, the system 2 can be placed within a number of monitoring and treatment configurations, with chosen monitoring system 50. Several representative configurations are shown at FIGS. 6, 6a, 6b, and 6c.

Turning to FIG. 6, the monitoring system 50 has a computerized device, in this embodiment in the form of a laptop 62. The laptop interfaces with the patient through a keyboard 60 and monitoring screen 52. The monitoring system 50 can interface with the patient through any means known in the art, such as, for example, a keyboard, touch screen, wireless audio inputs, or other control method known in the art.

A scanner 54 is at least one scanner and can be a single scanner or multiple scanners and take any appropriate readings. The at least one scanner can take a number of readings of the patient and send the data to the at least one computerized device 12. Scanned readings can include, for example, scanning for signs and symptoms, such as, e.g., pulse, blood pressure, and respiration. The at least one scanner 54 can include other scanning devices, such as a camera and visual scanning apparatus. The visual appearance can scan and monitor such things as pupil dilation and skin condition or injury.

Further, a temperature monitor 56 can monitor the room (i.e., ambient) temperature, the patient's temperature, or both, and track the difference. It particularly can track the patient temperature as a health parameter. An alarm 64 can alert the user or patient if a health parameter, such as temperature, pulse, etc, falls too far outside of what the system 2 considers normal (within allowable parameters).

Turning to FIG. 6a, a wall mounted unit 66 is shown. This embodiment also includes an instrument holding/connection device 58 configured to hold, support, or connect other monitoring instruments to the system 2. The device can support or connect other equipment, in a modular fashion, that might not normally be included in the system 2. For example, the device 58 can support a heart monitor to monitor a patient's heart condition. Or, for further examples, the device 58 may connect and integrate biometric devices, or devices such as an EKG machine, a CAT scanner, an MRI machine, or any diagnostic instrument. It may also, as another example, incorporate a brain wave monitor for a patient or user with epilepsy, to predict and control seizures.

Turning to FIG. 6b, a wall-mounted unit with stand 68 is shown.

Turning to FIG. 6c, a mobile robotic unit 70 is shown. The robotic unit 70 can be particularly useful when the user is unable to come to the monitoring unit 50. The robotic unit 70 can come to the user and take readings.

### Example 6

Allie's grandmother, Rebecca, is somewhat bedridden and being monitored 20 by the system 2. Allie wants to keep her at home if possible, and out of a nursing home, but also wants to be able to maintain a proper level of care. Rebecca, only being able to get out of bed a limited amount, is prone to bedsores. Bedsores, if not found and treated, can enlarge and become infected. They can become a health risk, and if prevalent enough, the patient may be removed from the home by health authorities.

The system 2 monitoring Rebecca has a camera and other visual scanning equipment as part of its at least one scanner 54. This can provide a regular visual scan of Rebecca and check for bedsores. If any bedsores appear during monitoring 20, the system can move into regular treatment 30 and determine a treatment plan 42 to be implemented 44. Allie can implement treatment or have an employed caregiver do so.

If the bedsores become large enough or infected, the system 2 can recognize this point and recommend professional medical treatment 40.

This way, Allie can be secure that this condition is being monitored for, can be treated most of the time by her or other caregivers, and if it becomes serious, she will be notified.

Those who can benefit from this system 2 include, for a few examples, those wanting to maximize personalization of their healthcare and minimize their worries, homebound people, caregivers, home health nurses, nurse practitioners, and individuals who want easy access to medical advice and consultation, people with conditions needing treatment who need to call their doctor or nurse and be triaged by the doctor or nurse, and people who experience a medical emergency at home and need instructions on procedures, such as CPR.

As has been shown, this system 2 can provide early detection and intervention. The system's 2 AI can monitor various health parameters and detect abnormalities or early signs of potential health issues. This can enable early intervention and treatment, leading to better health outcomes. For example, a smart device that tracks blood pressure can alert patients to hypertension trends and prompt them to seek medical attention or make lifestyle changes.

The system 2 further provides increased accessibility and convenience. The system 2 brings healthcare monitoring and assessment to the comfort of the user's home, reducing the need for frequent visits to healthcare facilities. This is particularly beneficial for individuals with limited mobility, those living in remote areas, or those who have difficulty accessing healthcare services due to various barriers.

The system 2 further provides personalized health insights: The AI algorithms of the system 2 can analyze data collected from users and provide personalized health insights and recommendations. Users can gain a better understanding of their own health status, track progress overtime, and make Informed decisions about their ongoing care. For instance, if a patient has sleep problems, a device that monitors sleep patterns can be added and offer personalized suggestions for Improving sleep quality.

The system also provides patients and users with empowerment and self-management. By using the AI smart medical devices of the system, individuals can take an active role in their healthcare and self-management. They can monitor their own vital signs, track symptoms, and engage in preventive measures, such as healthy lifestyle choices or medication adherence. This empowers users to be proactive about their health and potentially improve their overall well-being.

There is also a cost savings with the system 2. Regular monitoring with the system 2 can help identify health Issues early on, potentially reducing the need for expensive medical interventions or hospitalizations. Additionally, remote monitoring and virtual consultations facilitated by the devices of the system 2 can save costs associated with travel and in-person healthcare visits.

Health education and awareness can also be increased. The system 2 comes with user-friendly interfaces and can also come with educational resources that provide valuable health information and promote health awareness. The user can learn about their conditions, symptoms, treatment options, and preventative measures. This promotes health literacy and encourages individual users to make informed decisions regarding their health.

The system 2 can, overall, provide reassurance and peace of mind for users, as well as caregivers and family members who are responsible for monitoring the health of their patients or loved ones. The system 2 can offer continuous monitoring and alerts, enabling caregivers to respond quickly in case of emergencies or changes in health status.

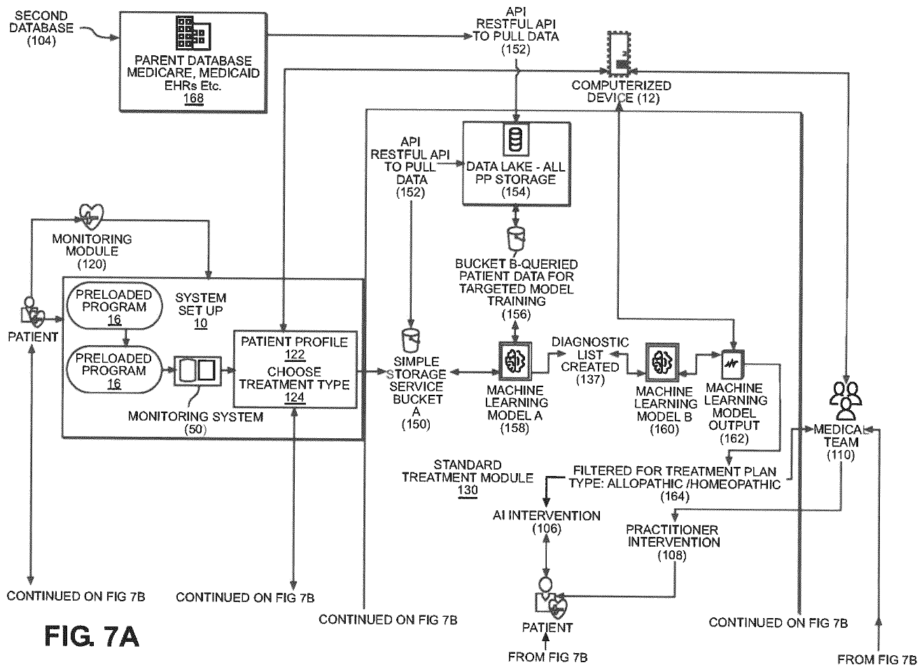

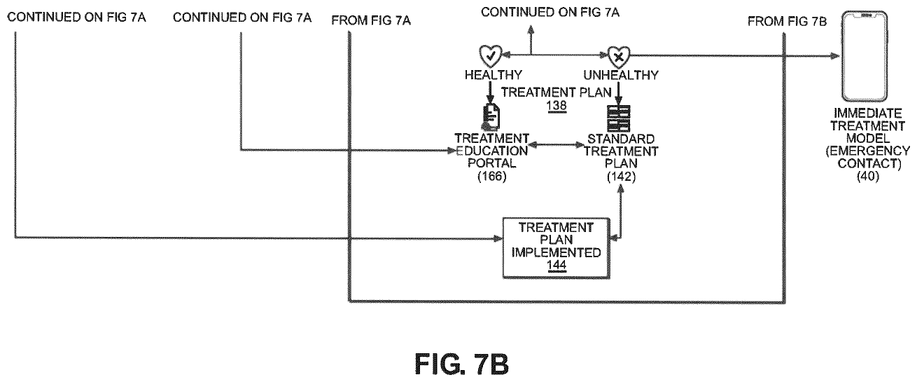

Turning to FIG. 7, one possible embodiment featuring the AI and equipment of the system 2 in use is shown.

A patient is monitored via a monitoring system 50 with the equipment therein connected to at least one computerized device 12. The computerized device 12 can be a virtual, and/or cloud or physical server. The server 12 with the pre-programmed software 16 allows a user access to build a patient profile 122 with the system 2. The at least one computerized device 12 can also be a server connected to at least one more portable computerized device 12a.

The patient can choose allopathic or homeopathic treatment 124 types as a part of the patient profile portal 122, or before or after building the patient profile 122, so long as this is chosen before treatment 130.

Generally, the patient Is continuously monitored via the Monitoring Module 120 as depicted. It is noted that the Monitoring Module 120 represents a specific version of the general Monitoring Module 20, the patient profile 122 represents a specific version of the patient profile built in 22. Likewise, choosing an allopathic or homeopathic treatment 124, the Treatment Module 130, the Diagnosis Phase 131, the Diagnostic Output 137, the Treatment Phase 138, determining the List of Treatment Plans 142, and implementing the Treatment Plan 144, represent specific versions of respective general steps 24, 30, and 31, 37, 38, 42 and 44.

This data can be accessed as needed by the diagnostic and treatment module 130 and, if utilized, a reviewing medical team 110 via the patient profile portal 122.

The collected data from monitoring the patient 120 can be transferred from at least one server 12 to a storage area; in this embodiment a Storage Bucket A 150 by restful API 152.

If monitoring uncovers a possible reading outside of normal range (i.e., symptom), the system 2 can proceed to the treatment module 130. The system 2 will begin the Diagnostic Phase 131 by receiving the data from Storage Bucket A 150 to Machine Learning Model A 158, Machine Learning Model B 160, or both. A storage bucket Is defined as a containerized system used for storing and managing vast amounts of unstructured data.

Machine Learning Model A 158 will be capable of assessing patient data and providing suggested diagnoses according to identified trends and scientific understandings.

Machine Learning Model B 160 will be capable of assessing patient diagnosis and provide suggested treatments according to identified medical trends and scientific understandings.

Data from Storage Bucket A 150 will additionally be requested by API 152 to be stored in a Data Lake 154. The Data Lake 154 is generally a specific form and embodiment of the first database 102 (see FIG. 4). The Data Lake 154 serves as a centralized repository to store vast amounts of structured, semi-structured, and unstructured data.

Data from a parent database 168, such as, e.g., Medicare or Medicaid database, can be downloaded into a suitable storage area such as a Data Lake 154. The parent database 158 is generally a specific form and embodiment of the second database 104 (see FIG. 4). Data from the parent database 168 can be requested by the system, or sent from the parent database 168.

The Machine Learning Model A 158 will have the capability of requesting all data relevant to the target patient(s) for comparative analysis of current data (Storage Bucket A 150) and historical health data (Another storage area; in this embodiment Storage Bucket B 156) related to diagnosis and treatment. The Diagnostic Output 137 created from the Machine Learning Model A 158 will be a list of possible diagnoses that will serve as input for Machine Learning Model B 160. Machine Learning Model B 160 will ingest the diagnosis to begin treatment phase 138 and determine and give a list of treatment plans 142 known in the art based on the condition and demographics of the patient. The treatment plan options will be filtered for either allopathic or homeopathic treatment 164, based on previous input in the patient profile.

Once decided upon, the combined list of possible diagnoses and treatments via Machine Learning Model Output 162 will be uploaded to the patient profile for review. The user will then be able to access the treatment and diagnosis and implement the treatment 144. The user will be offered access to Treatment Education Portal 166, which will include a detailed description of the diagnosis, treatments, and proper medication handling.

Before treatment begins, the diagnostic and treatment information can be sent to the medical team 110 for review. The medical team 110 can be any group of healthcare professionals of one or more who collaborate to provide patient care and medical services within the scope of the system 2. A medical team 110 may be composed of but not limited to physicians (e.g., primary care doctors, specialists, surgeons), nurses, therapists (e.g., physical therapists, occupational therapists), pharmacists, and other allied health professionals (e.g., medical assistants, dietitians).

The medical team 110 can provide a human review to ensure evidence-based comparison of diagnostic or treatment output from the system 2 to their own knowledge and experience. The medical team provides a level of human-based review as part of creating an effective treatment plan. The medical team 110 can prioritize patient welfare and ensure ethical decision-making in all aspects of care delivery, superseding system 2 as a safeguard for patient safety. Medical teams adhere to ethical principles and legal standards in their practice, including patient confidentiality, informed consent, privacy regulations (e.g., HIPAA), and professional codes of conduct.

Further, the medical team110 can actively participate in quality improvement initiatives aimed at enhancing patient safety, improving clinical outcomes, and optimizing healthcare delivery processes. The medical team 110 can analyze performance metrics, identify areas for improvement, and implement evidence-based interventions to drive positive changes. The medical team can also work with data scientists and engineers to improve system efficiency and accuracy.

The system and/or the medical team 110 can decide the condition requires Immediate emergency care and act appropriately. The medical team 110 can also agree with the AI-supplied diagnosis and treatment recommendations and pass this agreement on. The system 2 may deem the patient to be unhealthy and in need of treatment, recommend the treatment, and continue monitoring of the patient. If the treatment is successful, it will eventually end. If it is not successful, the process can begin again, seeking a more appropriate diagnosis.

Upon a medical team 110 review, there can be an AI intervention 106 or practitioner intervention 108 to stop or alter the system-recommended treatment. The form of AI intervention 106 can be dependent upon the type of allopathic or homeopathic path chosen 124. There can also be a direct practitioner intervention 108. The medical team 110 can also review or make a recommendation or determination regarding a homeopathic or allopathic path 124.

Further, the patient can also review the data and progress via a treatment educational portal 166, which will increase the overall amount of information available to the patient.

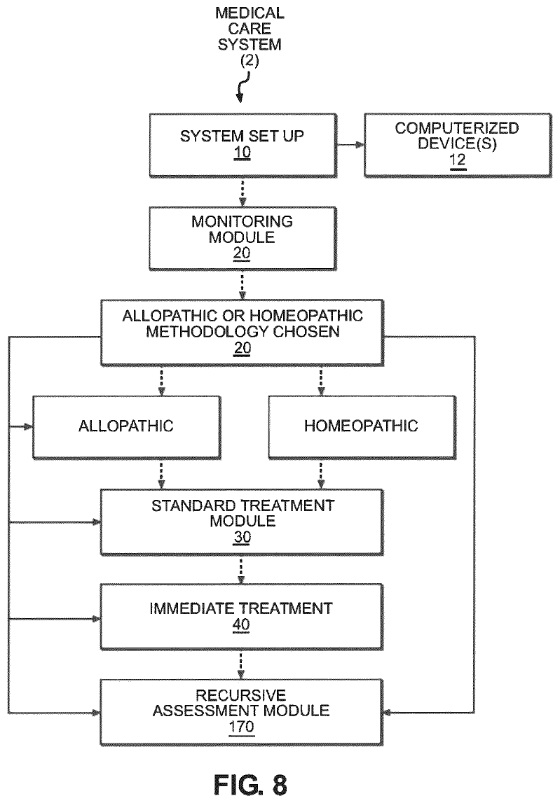

Turning to FIG. 8, a recursive assessment phase 170 can also be added to the process of the system 2. In this phase 170, the AI of the system can compare data such as treatment and results data from the patient, to other iterations of the same data for the same patient, to data from other patient files, or both. Over a number of comparisons and processing by the AI, predictions, treatments, and results can be recursively improved over time. The more data is added, especially with machine learning, the more monitoring, diagnosis, and treatment plans for the patient will Improve going forward.

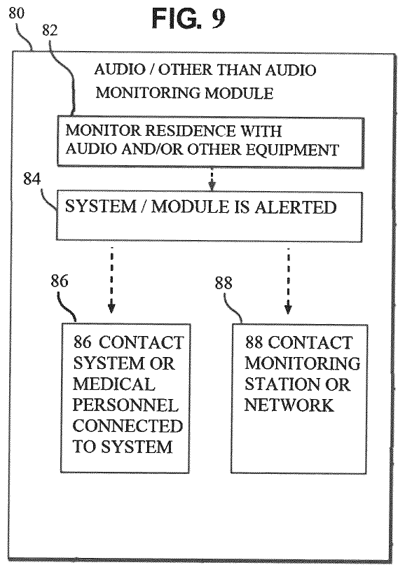

In another embodiment, and as shown in FIG. 9, an interactive audio/visual module 80 can also be added to the system 2. The interactive module 80 can assist with the safety, for example, of a resident living at home. The module 80 can be comprised of detection equipment for monitoring a residence. The equipment can be audio detection equipment, visual, vibration, or motion detecting equipment, or other suitable equipment known in the art for the purpose herein. In other embodiments, at least one biosensor, configured to monitor at least one chemical or biological reaction of a user, can be linked to the system 2, and can comprise some or all of the detection equipment.

The equipment 82 is Installed and monitored by the system 2. If, for example, a sufficiently loud sound or disturbance is detected by the system 2, it can become alerted 84. Or if a resident is on the floor for a designated amount of time, a visual equipment and system 2 may become alerted this way 84. Or if a biosensor picks up an abnormal change in one or more bio-sensor readings, it may become alerted 84. The system 2, via the interactive module 80 can inquire after the user, or can require input to prevent an alarm. If the system 2 or module 80 does not receive this input, it can alert friends, relatives, a doctor's office, or call emergency services, or take other appropriate action.

The system 2 can contact whatever personnel it has been programmed to contact in such situations 88, including emergency services, relatives or friends, a doctor, or other medical personnel. In other embodiments, the system 2 can contact a monitoring station or network with the Information 88, and from there, the monitoring station or network may contact the user, dispatch personnel of their own, or contact emergency services, or some combination.

### Example 7

June chooses to live alone, and her children are concerned because of her advanced age or conditions. Along with the system, audio equipment 82 is installed at June's home as a component of an audio monitoring module 80. If June passes out, falls, yells out, or a disturbance is otherwise created, the audio or visual monitoring equipment 82 or other equipment can pick it up. The system 2 is alerted 84 and requests that June let it know that she is well. Unfortunately, June does not do so. The system 2, as programmed, contacts June's children 86. When one of them does not get an answer on the phone, she calls 911 and help is immediately sent to June. It turns out June fell down, and she is looked after. When returned to her home, June and her children know she can be home by herself yet be looked after.

### Example 8

Henry lives alone, so he has Installed the system 2, Including an interactive monitoring module 80, and made it a point to wear a biosensor as detecting equipment 82. Henry passes out, which includes major changes in biological signs, including heartbeat and pulse. The biosensor 82 becomes alerted to the changes. The system 2 requests that Henry let it know that he is well. Henry does not do so. The system 2, as programmed, contacts a monitoring station which is part of an independent network that monitors multiple residents. The network sends its own medical person and calls 911 services, so help is immediately sent to Henry. Henry is on his way to the hospital within minutes and is saved.

It is to be understood that while certain forms of the present invention have been Illustrated and described herein, the expression of these Individual embodiments is for illustrative purposes and should not be seen as a limitation upon the scope of the invention. It is to be further understood that the invention is not to be limited to the specific forms or arrangements of parts described and shown.

1. A method of providing an artificial intelligence based home health system, comprising the steps of:
providing a setup module configured to set up the initial requirements of the system,
wherein the setup module is further comprised of the step of providing at least one computerized device,
providing a monitoring module configured to monitor at least one condition of at least one user,
providing at least one diagnostic possibility,
providing a treatment module configured to assist in carrying out basic treatment,
and providing an immediate treatment module configured to become alerted when the user needs Immediate medical treatment.

2. A method of providing an artificial intelligence based home health system, according to claim 1, wherein the system setup module is further comprised of the steps of:
providing system software configured to perform the functions of the system, and
loading the system software into the at least one computerized device.

3. A method of providing an artificial Intelligence based home health system, according to claim 1,
wherein the monitoring module is further comprised of the steps of:
creating a patient profile of collected data regarding the at least one user to be monitored,
and continuously monitoring the at least one user.

4. A method of providing an artificial intelligence based home health system according to claim 1, further comprising the step of choosing between
an allopathic treatment path,
a homeopathic treatment path,
or a combination of these.

5. A method of providing an artificial intelligence based home health system according to claim 1, wherein the treatment module is further comprised of
a diagnostic phase, wherein the diagnostic phase is comprised of the step of generating a list of at least one probable diagnosis,
and a treatment phase, wherein the treatment phase is comprised of providing a treatment plan from the list of at least one probable diagnosis.

6. A method of providing an artificial intelligence based home health system according to claim 1, further comprising the steps of:
detecting when at least one user condition falls outside a normal range, wherein the normal range is determined by the system, and
recommending whether the user either;
treat the at least one condition within the treatment module, or
move to the immediate treatment module for Immediate treatment.

7. A method of providing an artificial intelligence based home health system according to claim 1, wherein there is more than one diagnostic possibility, and further comprising the steps of
determining an order of treatment based on the order of likelihood of each diagnosis, and
is determining whether multiple diagnostic possibilities can be treated simultaneously.

8. A method of providing an artificial Intelligence based home health system comprising the steps of:
providing a system setup module configured to set up the system,
wherein the system set up module is comprised of the steps of;
providing at least one computerized device, and
providing system software configured to operate the system,
and loading the system software into the at least one computerized device providing a monitoring module comprised of the steps of;
creating a patient profile that includes data of at least one user to be monitored,
continuously monitoring the at least one user,
detecting when the at least one condition falls outside a normal range as determined for the at least one user by the system,
providing a treatment module and immediate treatment module,
and recommending whether to;
treat the user within the treatment module,
or
move to the immediate treatment module.
wherein the treatment module is further comprised of;
a diagnostic phase comprising generating a list of at least one probable diagnosis,
and a treatment phase comprising providing a treatment plan.

9. A method of providing an artificial intelligence based home health system, according to claim 8,
wherein the monitoring module is further comprised of the step of:
providing continuous re-assessment by the system of the results of monitoring during continuous monitoring of the at least one user.

10. A method of providing an artificial intelligence based home health system, according to claim 8, comprising the further steps of:
providing at least a first patient database,
transferring past patient data, current patient data, or both of a user into the at least a first patient database.
wherein the at least first patient database has at least one other patient profile for comparison.

11. A method of providing an artificial intelligence based home health system, according to claim 8, providing the further step of:
signaling the level of care the system has determined the at least one user needs at continuously during the continuous monitoring,
wherein the signaling is an audio signal, colored light set, printout, text, or a combination of these.

12. A method of providing an artificial intelligence based home health system, according to claim 8,
further comprising a recursive assessment phase, comprising the steps of, comparing current data of the at least one user to;
at least one other iterations of the same data for the at least one other user,
to data from other patient files,
or both.

13. A method of providing an artificial intelligence based home health system according to claim 8, wherein the treatment module comprises the further steps of:
determining whether at least one condition of the at least one user is moving closer to, or further from, an pre-determined acceptable range, and either;
moving back to continuous monitoring if the at least one condition moves back within an acceptable range,
or
moving to the immediate treatment module if the at least one condition moves sufficiently far outside the acceptable range.

14. A method of providing an artificial intelligence based home health system, according to claim 8, comprising the further steps of:
providing an interactive audio/visual module comprised of equipment for monitoring the safety of the at least one user,
wherein the equipment can be comprised of audio detection equipment, visual monitoring equipment, vibration detection equipment, motion detection equipment, or a combination of these,
installing the equipment,
monitoring readings from the equipment,
placing the system into an alert status if the system determines a sufficient disturbance has been detected by the equipment,
and contacting either:
at least one outside party,
a monitoring station.
or a monitoring network.

15. A method of providing an artificial intelligence based home health system according to claim 8, further comprising the step of:
providing at least one reason for each probable diagnosis on the list of at least one probable diagnosis.

16. A method of providing an artificial intelligence based home health system, according to claim 8, comprising the further step of:
providing either
a medical team comprised of at least one medical professional,
a medical practitioner,
or a combination of these,
and providing a review by the medical team, practitioner, or both of the list of at least one probable diagnosis, treatment plan, or both.

17. A method of providing an artificial intelligence based home health system, comprising the steps of:
providing at least one computerized device,
providing system software to the at least one computerized device, wherein the system software is configured to operate the home health system,
collecting data of at least one user and constructing at least one patient profile of the at least one user,
providing a monitoring system connected to the at least one computerized device,
determining a normal range of at least one biological condition of the at least one user based on data in the patient profile,
continuously monitoring the at least one patient with the monitoring system to continuously determine whether the at least one biological condition stays within the determined normal range,
providing a Diagnostic Phase comprised of the step of;
providing at least one diagnostic possibility if the continuous monitoring reveals a reading outside of the determined normal range for the at least one biological condition,
and providing a Treatment Phase comprised of the step of;
recommending at least one treatment if the at least one diagnostic possibility is provided.

18. A method of providing an artificial intelligence based home health system, according to claim 17, comprising the further steps in the Diagnostic Phase of:
transferring at least a portion of the collected data of the at least one user from the at least one computerized device to a storage area,
transferring data from the storage area to a first Machine Learning Model, a second Machine Learning Model, or both,
and wherein the at least one diagnostic possibility Is provided with the first Machine Learning Model.

19. A method of providing an artificial intelligence based home health system, according to claim 17, further comprising the steps of:
providing an immediate treatment module comprising the steps of:
determining that the at least one condition requires immediate emergency care, wherein the determination is made by
the system,
at least one medical professional,
or both.

20. A method of providing an artificial intelligence based home health system, according to claim 17, comprising the further steps of:
providing diagnostic data or at least one probable diagnosis form a parent database outside the system,
wherein the diagnostic data or at least the one probable diagnosis is either
sent by the parent database to the system, or
requested by the system of the Parent Database and sent from the Parent Database to the system.

21. A method of providing an artificial intelligence based home health system, comprising the steps of:
providing at least one computerized device with software to operate the system,
providing a monitoring module configured to monitor at least one condition of at least one user,
providing a treatment module configured to assist in carrying out basic treatment,
wherein the basic treatment does not require use of medical facilities or a prescription.

22. A method of providing an artificial intelligence based home health system, according to claim 1, further comprising the steps of:
providing a system setup module;
wherein the system set up module is further comprised of the step of
providing at least one computerized device with system software,
providing a monitoring module comprised of the steps of:
continuously monitoring at least one user,
detecting when at least one user condition falls outside a normal range,
if the at least one user condition falls outside a normal range, recommending either;
treating the at least one condition within a treatment module
or,
moving to an immediate treatment module.
